(19) United States
(12) Patent Application Publication
(10) Pub. No.: US 2025/0144092 A1
(43) Pub. Date: May 8, 2025

(54) TREATMENT OF PSYCHIATRIC OR NEUROLOGICAL DISORDERS IN PATIENTS WITH PROMINENT ANHEDONIA USING IRDABISANT AS A MONOTHERAPY OR IN COMBINATION WITH AN ANTIPSYCHOTIC AGENT

(71) Applicant: Alto Neuroscience, Inc., Mountain View, CA (US)

(72) Inventors: Amit ETKIN, Mountain View, CA (US); Adam SAVITZ, Mountain View, CA (US); Nicholas Cooper, Mountain View, CA (US)

(21) Appl. No.: 18/933,579

(22) Filed: Oct. 31, 2024

Related U.S. Application Data
(60) Provisional application No. 63/596,965, filed on Nov. 7, 2023.
(60) Provisional application No. 63/631,301, filed on Apr. 8, 2024.

(51) Int. Cl. A61K 31/501; A61K 31/519; A61K 45/06; A61P 25/24

(52) U.S. Cl. CPC A61K 31/501; A61K 31/519; A61K 45/06; A61P 25/24

(57) ABSTRACT

This invention relates to the treatment of a psychiatric or neurological disorder in a patient in need thereof who has anhedonia (lack of pleasure or motivation to pursue rewarding activities) by administering irdabisant or a pharmaceutically acceptable salt thereof, either as a monotherapy, or in combination with an antipsychotic agent. Examples of such psychiatric or neurological disorder include major depressive disorder, bipolar depression (such as bipolar I or bipolar II disorders), post-traumatic stress disorder, substance use disorder, anhedonia-related aspects of schizophrenia (e.g., negative symptoms) and schizoaffective disorder (e.g., with mood and/or negative symptoms).

This application claims the benefit of U.S. Provisional Application Nos. 63/596,965, filed Nov. 7, 2023, and 63/631,301, filed Apr. 8, 2024, each of which is hereby incorporated by reference.

### FIELD OF THE INVENTION

This invention relates to the treatment of a psychiatric or neurological disorder in a patient in need thereof who has anhedonia (lack of pleasure or motivation to pursue rewarding activities) by administering irdabisant or a pharmaceutically acceptable salt thereof, either as a monotherapy, or in combination with an antipsychotic agent. Examples of such psychiatric or neurological disorder include major depressive disorder, bipolar depression (such as bipolar I or bipolar II disorders), post-traumatic stress disorder, substance use disorder, anhedonia-related aspects of schizophrenia (e.g., negative symptoms) and schizoaffective disorder (e.g., with mood and/or negative symptoms).

### BACKGROUND OF THE INVENTION

Clinical care for depression involves assessment and diagnosis based on a set of clinician-assessed and patient-reported symptoms, such as depressed mood, anhedonia (lack of experience of pleasure), diminished ability to think or concentrate, appetite changes, sleep and psychomotor changes, but not based on biological or quantitative behavioral variables. Notably, selection of an antidepressant medication is done purely by trial-and-error, with no symptom profile or biological or quantitative behavioral measures to inform medication choice. Typically, selective serotonin reuptake inhibitors (SSRIs) are selected as the first line treatment based on their generally better tolerability, but not because they are known to be more effective generally, nor more effective for that particular patient. Most patients, however, fail to respond adequately to the first medication, at which point selection of the next medication again follows a trial-and-error process. Indeed, it has been found that on average, failing one SSRI does not necessarily predict a different response to another SSRI versus an SNRI (a serotonin and norepinephrine reuptake inhibitor) or NDRI (a norepinephrine and dopamine reuptake inhibitor). See, e.g., Rush et al., N. Engl. J. Med., 354, 1231-1242, 2006. As such, typical clinical assessments do not provide information sufficiently useful for selection of subsequent medication trials, and therefore external information not available to the clinician is required for improving medication selection.

The economic, societal, and personal cost of depression is very large, with depression being the leading cause of disability worldwide. This is even more pronounced for treatment-resistant depression (see, e.g., Amos et al., J. Clin. Psychiatry, 79 (2), 2018), suggesting that finding the best medication for an individual patient early in the course of treatment would provide many downstream benefits to both the patient and society at large.

Similar clinical needs exist in other related conditions in which depressive symptoms exist. Such symptoms include low mood, lack of experience of pleasure or motivation to pursue rewarding activities (anhedonia), and impairments in attention, cognition or decision making. These conditions include major depressive disorder, bipolar depression (such as bipolar I or bipolar II disorders), post-traumatic stress disorder, substance use disorder, and depression-related or anhedonia-related aspects of schizophrenia (e.g., negative symptoms). Importantly, these different depression-related symptoms or areas of dysfunction co-occur and may be functionally related. For example, a patient with major depression may report depressed mood and anhedonia. Likewise, the same symptoms may be reported by patients diagnosed with other conditions in which similar impairments may co-occur, such as bipolar depression, post-traumatic stress disorder, or substance use disorder. Though schizophrenia is often thought of with respect to prominent hallucinations and delusions, the depression-like anhedonia-related negative symptoms are often the greater source of long-term disability and functional impairment. Hence, a treatment approach that encompasses these multiple and related functional systems would be both of importance to any one of these clinical conditions, and equally may be applicable across them.

Anhedonia symptoms, when present in patients with depressive symptoms, is a predictor of particularly poor outcome. Analysis of the landmark Sequenced Treatment Alternatives to Relieve Depression (STAR*D) study, for example, found that presence of anhedonia predicted poorer antidepressant treatment outcome even after controlling for overall depression severity (see, e.g., Uher et al., Psychol. Med., 42 (5), 967-980, 2012, PMID 21929846). Long-term outcomes are also worse for depressed patients with anhedonia compared to those without it (see, e.g., Spijker et al., Acta Psychiatry Scand., 103 (2), 122-130, 2001, PMID 11167315). Additionally, anhedonia is associated with poor psychosocial functioning (see, e.g., Vinckier et al., Eur. Psychiatry, 44, 1-8, 2017, PMID 28535406). In schizophrenia, there are no approved treatments for anhedonia or related negative symptoms. There are also no treatments currently approved by the U.S. Food and Drug Administration (FDA) targeted for the treatment of anhedonia in depression.

U.S. Pat. Nos. 8,207,168 and 8,673,916 describe certain pyridazinone derivatives and their use as H₃ inhibitors.

U.S. Publication No. 2022/0226331 describes the use of a combination of two or more of a D2 agonist, an adenosine A2A receptor antagonist, a histamine H₃ antagonist or inverse agonist, a mGluR5 receptor antagonist, or a nicotinic α₄-β₂ and/or az receptor agonist.

There is a need for new and improved methods for treating psychiatric and neurological disorders in patients who have anhedonia.

### SUMMARY OF THE INVENTION

In one aspect, the present invention relates to a method of treating a psychiatric or neurological disorder in a patient (e.g., a patient in need thereof) with anhedonia (i.e., a patient in which anhedonia or motivational function exists). The method comprises administering to the patient an effective amount of irdabisant or a pharmaceutically acceptable salt thereof as the sole active agent or in combination with one or more antipsychotic agents. In one embodiment, the psychiatric or neurological disorder is major depressive disorder, bipolar depression, post-traumatic stress disorder, substance use disorder, anhedonia-related aspects of schizophrenia, schizoaffective disorder, or any combination of any of the foregoing. In another embodiment, the psychiatric or neurological disorder is motivational deficits or anhedonia in schizophrenia. In yet another embodiment, the psychiatric or neurological disorder is depression.

The present inventors have also discovered that the Bond-Lader Visual Analogue Scale (BL-VAS) can be used as a measurement for ‘depressed mood’. One embodiment is a method for evaluating a drug for the treatment of depression (such as major depressive disorder) comprising, for each patient, (a) evaluating the patient prior to initiation of treatment with the drug using the BL-VAS, (b) initiating treatment of the patient with the drug, and (c) at least once evaluating the patient during treatment with the drug using the BL-VAS, wherein the drug is considered effective for depression if there is an improvement in the BL-VAS for the patients tested. In one embodiment, the drug is considered effective for depression if there is an improvement for the patients tested in the BL-VAS total score, BL-VAS alertness sub-score, BL-VAS calmness sub-score, BL-VAS contentedness, or any combination of any of the foregoing.

Another aspect is a method of treating anhedonia in a patient by administering to the patient an effective amount of irdabisant or a pharmaceutically acceptable salt thereof as the sole active agent (i.e., as a monotherapy) or in combination with one or more antipsychotic agents.

Another aspect is a method of treating anhedonia in a patient having major depressive disorder (MDD) by administering to the patient an effective amount of irdabisant or a pharmaceutically acceptable salt thereof as the sole active agent (i.e., as a monotherapy) or in combination with one or more antipsychotic agents, antidepressants, or any combination of any of the foregoing. In another embodiment, the antidepressants are selected from selective serotonin reuptake inhibitors (SSRI), serotonin and norepinephrine reuptake inhibitors (SNRI), atypical antidepressants, norepinephrine and dopamine reuptake inhibitor (NDRI), and any combination of any of the foregoing. In one embodiment, the antidepressants are not D2 agonists (such as D2/D3 agonists), adenosine A2A receptor antagonists, histamine H₃ antagonists or inverse agonists, mGluR5 receptor antagonists, or nicotinic α₄-β₂ and/or az receptor agonists.

In one embodiment of any of the methods described herein, the method comprises comprising administering to the patient an effective amount of irdabisant, or a pharmaceutically acceptable salt thereof (such as irdabisant hydrochloride), wherein the irdabisant, or a pharmaceutically acceptable salt thereof, is the sole active ingredient (i.e., as a monotherapy).

In another embodiment of any of the methods described herein, the method comprises administering to the patient an effective amount of irdabisant, or a pharmaceutically acceptable salt thereof (such as irdabisant hydrochloride), and an antipsychotic agent.

In one embodiment, the antipsychotic agent is a typical (first generation) antipsychotic, an atypical (second generation) antipsychotic, a third generation (D2 partial agonist) antipsychotic, or any combination of any of the foregoing.

In one embodiment, the antipsychotic agent is a typical (first generation) antipsychotic.

In one embodiment, the typical (first generation) antipsychotic agent is a butyrophenone (such as, e.g., benperidol, bromperidol, droperidol, haloperidol, and moperone), a diphenylbutylpiperidine (such as, e.g., fluspirilene, penfluridol and pimozide), a phenothiazine (such as, e.g., acepromazine, chlorpromazine, cyamemazine, dixyrazine, fluphenazine, levomepromazine, mesoridazine, perazine, periciazine, perphenazine, peoptiazine, prochlorperazine, promethazine, prothipendyl, thioproperazine, and trifluoperazine), a thioxanthene (such as, e.g., chlorprothixene, clopenthixol, flupentixol, thiothixene and zuclopenthixol).

In one embodiment, the antipsychotic agent is an atypical (second generation) antipsychotic.

In one embodiment, the atypical (second generation) antipsychotic agent is a benzamide (such as, e.g., amisulpride, nemonapride, remoxipride and sultopride), a benzisoxazole or benzisothiazole (such as, e.g., lumateperone, lloperidone, paliperidone, perospirone, risperidone, ziprasidone and lurasidone), a butyrophenone (such as, e.g., melperone and lumateperone), a tricyclic (such as, e.g., asenapine, clozapine, olanzapine, quetiapine and zotepine), blonanserin, pimavanserin, sertindole, or a pharmaceutically acceptable salt of any of the foregoing, or any combination of any of the foregoing.

In one embodiment, the third generation (D2 partial agonist) antipsychotic is a phenylpiperazine or quinolinone (such as, e.g., aripiprazole, brexpiprazole and cariprazine), a phenylpiperazine or benzoxazinone (such as, e.g., brilaroxazine), or a pharmaceutically acceptable salt of any of the foregoing, or any combination of any of the foregoing.

In one embodiment, the antipsychotic agent is risperidone, aripiprazole, brexpiprazole, amisulpride, olanzapine, quetiapine, or a pharmaceutically acceptable salt thereof.

In a preferred embodiment, the antipsychotic agent is risperidone, or a pharmaceutically acceptable salt thereof.

In one embodiment of any of the methods described herein, the psychiatric disorder is major depressive disorder, bipolar depression, post-traumatic stress disorder, substance use disorder, anhedonia-related aspects of schizophrenia (e.g., negative symptoms), schizoaffective disorder (e.g., with mood and/or negative symptoms) (for instance, schizoaffective disorder-bipolar type or schizoaffective disorder-depressive type), or any combination of any of the foregoing.

In one embodiment of any of the methods described herein, the psychiatric disorder is s bipolar I disorder or bipolar II disorder.

In one embodiment of any of the methods described herein, the psychiatric disorder is negative symptoms of schizophrenia.

In one embodiment of any of the methods described herein, the psychiatric disorder is motivational deficits or anhedonia in schizophrenia.

In one embodiment of any of the methods described herein, the psychiatric disorder is depression.

In one embodiment of any of the methods described herein, the irdabisant, or a pharmaceutically acceptable salt thereof (such as irdabisant hydrochloride) is not administered in combination with a D2 agonist (such as a D2/D3 agonist), an adenosine A2A receptor antagonist, a histamine H₃ antagonist or inverse agonist, a mGluR5 receptor antagonist, or a nicotinic α₄-β₂ and/or az receptor agonist, or any combination of any of the foregoing.

In one embodiment of any of the methods described herein, the irdabisant, or a pharmaceutically acceptable salt thereof (such as irdabisant hydrochloride) is not administered in combination with an adenosine A2A receptor antagonist, a histamine H₃ antagonist or inverse agonist, a mGluR5 receptor antagonist, or a nicotinic α₄-β₂ and/or az receptor agonist, or any combination of any of the foregoing.

### BRIEF DESCRIPTION OF THE DRAWINGS

FIG. 1 is a line plot of dopamine efflux in an in vivo micro dialysis experiment in the nucleus accumbens of rats, with dopamine levels post-dosing plotted as a percentage of baseline. Doses are intraperitoneal administration of 0.001, 0.01, 0.3 and 10 mg/kg of irdabisant. DA=dopamine.

FIG. 2 is a line plot of dopamine efflux in an in vivo micro dialysis experiment in the nucleus accumbens of rats, with dopamine levels post-dosing plotted as a percentage of baseline, in an experiment where either irdabisant was given alone (0.01 mg/kg IP), risperidone was given alone (0.1 mg/kg IP), or the two were given in combination. at the same doses.

FIG. 3 is a bar plot of the area under the curve analysis over the first 210 minutes post-dosing showing significantly greater dopamine release when irdabisant (0.01 mg/kg IP) was combined with risperidone (0.01 mg/kg IP) compared to either single treatment alone at the same dose. DA=dopamine, AUC=area under the curve.

FIGS. 4A and 4B show scatterplots of the correspondence between PHQ for a 1 week (FIG. 4A) or 2 week (FIG. 4B) delay following the baseline measurement of BL-VAS. The correlations are −0.55 and −0.53 respectively indicating that increasing ‘Contentedness’ (also referred to as ‘mood’) is predictive of lower PHQ scores (reduced depressive symptoms) 1 or 2 weeks subsequently. Both results are highly statistically significant (p<0.0001) and employ 170 and 169 pairs of observations from participants respectively.

### DETAILED DESCRIPTION OF THE INVENTION

Unless specifically stated or obvious from context, as used herein, the term “or” is understood to be inclusive.

Unless specifically stated or obvious from context, as used herein, the terms “a”, “an”, and “the” are understood to be singular or plural.

Ranges provided herein are understood to be shorthand for all of the values within the range.

Unless specifically stated or obvious from context, as used herein, the term “about” is understood as within a range of normal tolerance in the art, for example within 2 standard deviations of the mean. The term “about” can also be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Unless indicated otherwise, the term “irdabisant” (also known as CEP-26401) refers to 6-{4-[3-((R)-2-methyl-pyrrolidin-1-yl)-propoxy]-phenyl}-2H-pyridazin-3-one, which has the structure:

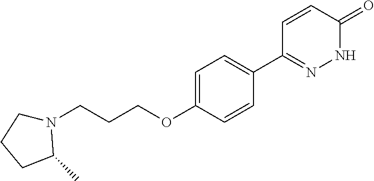 [SMILES: C[C@@H]1CCCN1CCCOc1ccc(-c2ccc(=O)[nH]n2)cc1]

Irdabisant and pharmaceutically acceptable salts thereof (such as the hydrochloride salt) can be prepared as described in U.S. Pat. No. 8,207,168, which is incorporated by reference in its entirety. A preferred salt of irdabisant is irdabisant hydrochloride.

Suitable antidepressants for concurrent therapy include, but are not limited to, (i) serotonin and norepinephrine reuptake inhibitors (SNRIs) (such as venlafaxine, duloxetine, milnacipran, sibutramine, SEP-227162, or LY 2216684), (ii) selective serotonin reuptake inhibitors (SSRIs) (such as escitalopram, fluoxetine, fluvoxamine, sertraline, citalopram, vilazodone, and paroxetine), (iii) atypical antidepressants (such as agomelatine, mianserin, mirtazapine, nefazodone, opipramol, tianeptine, and trazodone), and (iv) norepinephrine and dopamine reuptake inhibitors (NDRIs) (such as bupropion, amineptine, prolintane, dexmethylphenidate, and pipradrol). In one embodiment, the antidepressant is selected from SSRIs, SNRIs, mirtazapine, bupropion, or any combination of any of the foregoing. In another embodiment, the antidepressant is not a monoamine oxidase inhibitor (MAOI), a tricyclic antidepressant (TCA) (such as amitriptyline, imipramine, clomipramine, and desipramine), or ketamine. In one embodiment, the antidepressants are not D2 agonists (such as D2/D3 agonists), adenosine A2A receptor antagonists, histamine H₃ antagonists or inverse agonists, mGluR5 receptor antagonists, or nicotinic α₄-β₂ and/or az receptor agonists.

Pharmaceutically acceptable salts of the compounds described herein include, but are not limited to, those derived from the following acids as well as acid addition salts: mineral acids such as hydrochloric acid, sulfuric acid, phosphoric acid and sulfamic acid; and organic acids such as acetic acid, citric acid, lactic acid, tartaric acid, malonic acid, methanesufonic acid, ethanesulfonic acid, benzenesulfonic acid, p-toluenesulfonic acid, cyclohexylsulfamic acid, and quinic acid. The corresponding acid addition salts comprise the following: hydrohalides, such as hydrochloride and hydrobromide, sulfate, phosphate, nitrate, sulfamate, acetate, citrate, lactate, tartrate, malonate, oxalate, salicylate, propionate, succinate, fumarate, maleate, methylene-bis-B-hydroxynaphthoates, gentisates, mesylates, isethionates and di-p-toluoyltartratesmethanesulfonate, ethanesulfonate, benzenesulfonate, p-toluenesulfonate, cyclohexylsulfamate and quinate, respectively. In a preferred embodiment for irdabisant, the pharmaceutically acceptable salt is a hydrochloride salt.

The terms “treat,” “treatment,” and “treating” in the context of the administration of a therapy to a patient refers to the reduction or inhibition of the progression and/or duration of a disease or condition, the reduction or amelioration of the severity of a disease or condition, and/or the amelioration of one or more symptoms thereof resulting from the administration of one or more therapies.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, delay, inhibition, suppression, or reduction of a symptom or symptoms of a disease or disorder, which could also be referred to as a “therapeutically effective amount.” A “reduction” of a symptom or symptoms (and grammatical equivalents of this phrase) means decreasing of the severity or frequency of the symptom(s), or elimination of the symptom(s). An “effective amount” of a drug can be an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). Dosages may be varied depending upon the requirements of the patient and the compound being employed. The dose administered to a patient, in the context of the present disclosure, should be sufficient to effect a beneficial therapeutic response in the patient over time. The size of the dose may also be determined by the existence, nature, and extent of any adverse side-effects. Determination of the proper dosage for a particular situation is within the skill of the practitioner.

As used herein, the term “subject” or “patient” refers to an animal, such as a mammal, for example a human. The methods described herein can be useful in both human therapeutics and veterinary applications. In some embodiments, the patient is a mammal, and in some embodiments, the patient is human.

### Treatment

From a neurobiological perspective, anhedonia is hypothesized to arise from dysfunction in dopamine-related signal processing within the reward system (which includes the ventral tegmental area and the nucleus accumbens) as well as interconnected brain structures such as the anterior cingulate (see, e.g., Lambert et al., CNS Neurosci. Ther., 24 (7), 615-623, 2018, PMID 29687627, and Argyropoulos et al., J. Psychopharmacol., 27 (10), 869-877, 2013, PMID 23904408). Consistent with the important role of reward system dopamine in driving reward-based learning, depressed patients with anhedonia have deficits in reward-based learning, which in turn predicts worse treatment outcome (see, e.g., Vrieze et al., Biol. Psychiatry, 73 (7), 639-645, 2013, PMID 23228328). Likewise, dysfunction in the reward system, as well as in reward-based learning and decision making has been demonstrated in schizophrenia (see, e.g., Saleh et al., Front. Behav. Neurosci., 15, Article 709753, 2021, doi: 10.3389/fnbeh.2021.709753, PMID 34566594).

Without wishing to be bound by theory, the inventors theorize that, given the prominent role of reduced reward system dopaminergic release and signaling in anhedonia, a drug that increases dopamine release in areas with reduced dopamine release or dopamine-related signal processing, such as the nucleus accumbens, may be a particularly effective treatment for depression in patients with anhedonia. One such candidate mechanism involves the histamine H₃ receptor. This receptor is present only in the brain, and acts to inhibit the release of multiple neurotransmitters, including dopamine. As a receptor that is tonically active at baseline, which increases its activity further in response to binding of its ligand histamine, a drug that acts as an H₃ inverse agonist may act to disinhibit dopamine release in the reward system (principally, the nucleus accumbens, where terminals from the ventral tegmental area release dopamine to drive reward-related behavior). Prior to the present invention, H₃ inverse agonists have never been tested in a clinical trial for the treatment of depression, or of motivational deficits or anhedonia in any other disorder in which they are prominent, in human patients. As reviewed by Ghamari et al., Pharmacol Ther., 2019, 200:69-84, PMID 31028835, studies of H₃ inverse agonists have focused on sleep and cognitive disorders (including cognitive manifestations of Alzheimer's disease, attention deficit hyperactivity disorder and schizophrenia). Despite the large number of trials on a wide range of specific compounds, the potential utility of H₃ inverse agonists on disorders in which anhedonia or motivational symptoms are prominent (such as depression) have not been examined in part because it has been thought that drugs with this mechanism do not lead to increases in dopamine release within the reward system—an important function underlying the construct of anhedonia/motivational impairments (see below). Moreover, though animal models of depression-like behavior have shown a response to H₃ inverse agonists (Qian et al., Front Psychiatry, 2022, 13:825591, PMID 36213905), such models are well-known not to be able to generalize to effective therapeutics in humans with depression (Stanford, Altern Lab Anim., 2020, 48 (3):106-115, PMID 32777937), which may in part explain why many disorders have been tested for response to H₃ inverse agonists, but conditions such as depression have not.

Authors though have noted with surprise that H₃ inverse agonists (or antagonists) do not appear to increase dopamine release in the nucleus accumbens (Ellenbroek et al., Current Pharm. Des., 21 (26), 3760-3770, 2015, PMID 26044979). They suggested that H₃ inverse agonists may only increase dopamine release under conditions of high histamine release, and thus not at basal levels. This view is supported by data on the only FDA-approved H₃ inverse agonist, pitolisant, which is approved for excessive daytime sleepiness in narcolepsy patients due to its sleep arousing effects. It has been shown in striatal microdialysis experiments, that there is no increase in release of dopamine release in the nucleus accumbens with pitolisant, with the dopamine releasing drug modafinil serving as a positive control (Uguen et al., Br. J. Pharmacol., 169 (3), 632-44, 2013, PMID 23472741). A similar lack of effect on dopamine release in the striatum/nucleus accumbens has been observed for other H₃ inverse agonists such as ABT-239 ((R)-4-(2-(2-(2-methyl-|-pyrrolidinyl)ethyl)-1-benzofuran-5-yl)benzonitrile) and JNJ-39220675 ((4-cyclobutyl-1,4-diazepan-1-yl) (6-(4-fluorophenoxy)pyridin3-yl) methanone) (Fox et al., J. Pharmacol. Exp. Ther., 313 (1), 176-90, 2005, PMID 15608077, and Galici et al., Psychopharmacology (Berlin), 214 (4), 829-41, 2011, PMID 21086115).

Despite these negative data, there is precedent that different H₃ inverse agonists have discrepant effects on dopamine release in other parts of the brain. For example, while most H₃ inverse agonists increase dopamine release in the cortex, some, like JNJ-10181457 (4-[3-[4-[piperidiny]]but-1-ynyl]benzyl]morpholine dihydrochloride), do not (Ellenbroek et al., supra, and Bonaventure et al., Biochemical Pharmacology., 73 (8), 1084-1096, 2007, PMID 17129577). Unlike pitolsant, ABT-239, and JNJ-39220675, it has been discovered that irdabisant increases dopamine release in the nucleus accumbens.

Clinically, anhedonia can be assessed by scales such as, for example, the Snaith-Hamilton Pleasure Scale (SHAPS) and the Dimensional Anhedonia Rating Scale (DARS). Suitable scales for clinically assessing anhedonia are shown in the table below. In one embodiment, patients are assessed using the Positive Valence Systems Scale (PVSS).

| Scale | Exemplary Citations |
|---|---|
| Snaith-Hamilton Pleasure Scale | Snaith et al., Br. J. Psychiatry, 167, 99-103, |
| | 1995 |
| Dimensional Anhedonia Rating Scale | Rizvi et al., Psychiatry Res., 229, 109-19, |
| | 2015 |
| Positive Valence Systems Scale | Khazanov et al., Assessment, 27, |
| | 1045-1069, 2020 |
| Chapman Social Anhedonia Scale | Chapman et al., J. Abnorm. Psychol., 85, |
| | 374-382, 1976 |
| Chapman Physical Anhedonia Scale | Chapman et al., J. Abnorm. Psychol., 85, |
| | 374-382, 1976 |
| Temporal Experience of Pleasure Scale | Gard et al., Journal of Research in |
| | Personality, 40, 1086-1102, 2006 |
| Fawcett-Clark Pleasure Capacity Scale | Fawcett et al., Arch. Gen. Psychiatry, 40, 79- |
| | 84, 1983 |
| Motivation and Pleasure Scale-Self Report | Llerena et al., Compr. Psychiatry, 54, 568- |
| | 574, 2013 |
| Anticipatory and Consummatory | Gooding et al., Psychiatry Res., 215, 237- |
| Interpersonal Pleasure Scale | 243, 2014 |
| Motivation and Energy Inventory | Fehnel et al., Qual, Life Res., 13:1321-3136, |
| | 2004 |
| | Hewett et al., J. Psychopharmacol., 23, 531- |
| | 538, 2009 |
| Apathy Evaluation Scale | Marin et al., Psychiatry Res., 38,143-162, |
| | 1991 |
| Specific Loss of Interest Scale | Winer et al., J. Affect. Disord., 152-154, |
| | 193-201, 2014 |
| Self-Assessment Anhedonia Scale | Olivares et al., Neurology, Psychiatry, and |
| | Brain Research, 12(3), 121-134, 2005 |
| Sensitivity to Reinforcement of Addictive | Goldstein et al., J. Psychopharmacol., 24(2), |
| and other Primary Rewards questionnaire | 257-266, 2010 |

In one embodiment of any of the methods described herein, mood or depression in a patient is assessed with the Bond-Lader Visual Analogue Scale (BL-VAS). The questionnaire for the BL-VAS presents visual scales with the with the following comparisons to the prompt, “How do you feel right now?: (1) Alert versus Drowsy, (2) Calm versus Excited, (3) Strong versus Feeble, (4) Muzzy versus Clear-Headed, (5) Well-Coordinated versus Clumsy, (6) Lethargic versus Energetic, (7) Contented versus Discontented, (8) Troubled versus Tranquil, (9) Mentally-Slow versus Quick-witted, (10) Tense versus Relaxed, (11) Attentive versus Dreamy, (12) Incompetent versus Proficient, (13) Happy versus Sad, (14) Antagonistic versus Amicable, (15) Interested versus Bored, and (16) Withdrawn versus Gregarious.

In one embodiment of any of the methods described herein, the dose of irdabisant, or a pharmaceutically acceptable salt thereof (based on the equivalent amount of irdabisant hydrochloride), e.g., for oral administration, ranges from about 1 μg to about 500 μg per day. In one embodiment, about 5 μg to about 250 μg of irdabisant or a pharmaceutically acceptable salt thereof (based on the equivalent amount of irdabisant hydrochloride) is orally administered daily. In one embodiment, about 25 μg of irdabisant or a pharmaceutically acceptable salt thereof (based on the equivalent amount of irdabisant hydrochloride) is orally administered daily. In another embodiment, about 75 μg of irdabisant or a pharmaceutically acceptable salt thereof (based on the equivalent amount of irdabisant hydrochloride) is orally administered daily. In yet another embodiment, about 5 μg of irdabisant or a pharmaceutically acceptable salt thereof (based on the equivalent amount of irdabisant hydrochloride) is orally administered daily. In yet another embodiment, about 10 μg of irdabisant or a pharmaceutically acceptable salt thereof (based on the equivalent amount of irdabisant hydrochloride) is orally administered daily. In yet another embodiment, about 15 μg of irdabisant or a pharmaceutically acceptable salt thereof (based on the equivalent amount of irdabisant hydrochloride) is orally administered daily. In yet another embodiment, about 20 μg of irdabisant or a pharmaceutically acceptable salt thereof (based on the equivalent amount of irdabisant hydrochloride) is orally administered daily. In yet another embodiment, about 25 μg of irdabisant or a pharmaceutically acceptable salt thereof (based on the equivalent amount of irdabisant hydrochloride) is orally administered daily. In yet another embodiment, about 5 μg to about 25 μg of irdabisant or a pharmaceutically acceptable salt thereof (based on the equivalent amount of irdabisant hydrochloride) is orally administered daily. In yet another embodiment, about 15 μg to about 25 μg of irdabisant or a pharmaceutically acceptable salt thereof (based on the equivalent amount of irdabisant hydrochloride) is orally administered daily. In one embodiment, the dose of irdabisant is administered once daily. In yet another embodiment, about 25 μg of irdabisant hydrochloride is orally administered once daily. In yet another embodiment, about 75 μg of irdabisant hydrochloride is orally administered once daily.

In one embodiment of any of the methods described herein, the daily dose of irdabisant, or a pharmaceutically acceptable salt thereof, is administered in one or more separate doses, such as once a day, twice a day, three times a day or four times a day.

Suitable doses of risperidone or a pharmaceutically acceptable salt thereof (based on the equivalent amount of risperidone free base) for use in any of the methods described herein, e.g., for oral administration, range from about 0.25 mg to about 16 mg per day. In one embodiment, for an adult patient, the daily dose of risperidone or a pharmaceutically acceptable salt thereof (based on the equivalent amount of risperidone free base) ranges from about 2 to about 16 mg. In another embodiment, e.g., for an adult patient, the daily dose of risperidone or a pharmaceutically acceptable salt thereof (based on the equivalent amount of risperidone free base) ranges from about 1 to about 8 mg. For example, the daily dose of risperidone or a pharmaceutically acceptable salt thereof (based on the equivalent amount of risperidone free base) may be 1, 2, 4, 6, or 8 mg. In one embodiment, the amount of risperidone (or pharmaceutically acceptable salt thereof) is not titrated up (for example, no titration up occurs during initial treatment).

In one embodiment of any of the methods described herein, administration is by the oral route once a day (q.d.) or twice daily (b.i.d.). In one embodiment of any of the methods described herein, administration is by the oral route once a day (q.d.). In another embodiment of any of the methods described herein, oral administration is by a sustained release or extended release oral formulation.

### Pharmaceutical Compositions

Pharmaceutical compositions for use in any of the methods described herein can include one or more pharmaceutically acceptable excipients in addition to the active ingredient(s). Such pharmaceutical compositions may be suitable for any route of administration, such as, e.g. nasal, rectal, intercisternal, buccal, intramuscular, intrasternal, intracutaneous, intrasynovial, intravenous, intraperitoneal, intraocular, periosteal, intra-articular injection, infusion, oral, topical, inhalation, parenteral, subcutaneous, implantable pump, continuous infusion, gene therapy, intranasal, intrathecal, intracerebroventricular, transdermal, or by spray, patch or injection. In one embodiment, pharmaceutical compositions for use in any of the methods described herein are suitable for oral administration.

The pharmaceutical composition may be formulated as a solid dosage form, such as capsules, pills, soft-gels, tablets, caplets, troches, wafer, sprinkle, or chewing for oral administration. The pharmaceutical composition may also be formulated as a liquid dosage form such as an elixir, suspension or syrup.

The pharmaceutical composition may also be presented in a dosage form for transdermal application (e.g., a patch or an ointment) or oral administration.

The pharmaceutical composition may be in a liquid dosage form or a suspension to be applied to the nasal cavity or oral cavity using a dropper, a sprayer or a container. The pharmaceutical composition may be in a solid, salt or powder to be applied to nasal cavity or oral cavity using a sprayer, a forced air or a container.

Pharmaceutical compositions for use in any of the methods described herein may further comprise one or more pharmaceutically acceptable excipients, selected from, but not limited to, pharmaceutically acceptable carriers, binders, diluents, adjuvants, or vehicles, such as preserving agents, fillers, polymers, disintegrating agents, glidants, wetting agents, emulsifying agents, suspending agents, sweetening agents, flavoring agents, perfuming agents, lubricating agents (such as magnesium stearate), acidifying agents, coloring agent, dyes, preservatives and dispensing agents, and any combination of any of the foregoing. Suitable pharmaceutically acceptable excipients are described in, e.g., the Handbook of Pharmaceutical Excipients, 6th Ed., Pharmaceutical Press and American Pharmaceutical Association (2009).

Pharmaceutically acceptable carriers are generally non-toxic to recipients at the dosages and concentrations employed and are compatible with other ingredients of the formulation. Non-limiting examples of pharmaceutically acceptable carriers include water, saline, dextrose solution, ethanol, polyols, vegetable oils, fats, ethyl oleate, liposomes, waxes polymers, including gel forming and non-gel forming polymers, and suitable mixtures thereof. The carrier may contain minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulin; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

Examples of binders include, but are not limited to, microcrystalline cellulose and cellulose derivatives, gum tragacanth, glucose solution, acacia mucilage, gelatin solution, molasses, polyvinylpyrrolidone, povidone, crospovidone, sucrose and starch paste.

Examples of diluents include, but are not limited to, lactose, sucrose, starch, kaolin, salt, mannitol and dicalcium phosphate.

Examples of excipients include, but are not limited to, starch, surfactants, lipophilic vehicles, hydrophobic vehicles, pregelatinized starch, microcrystalline cellulose, lactose, milk sugar, sodium citrate, calcium carbonate, and dicalcium phosphate. Typical excipients for dosage forms such as a soft-gel include gelatin for the capsule and oils such as soy oil, rice bran oil, canola oil, olive oil, corn oil, and other similar oils; glycerol, polyethylene glycol liquids, and vitamin E TPGS as a surfactant.

Examples of disintegrating agents include, but are not limited to, complex silicates, croscarmellose sodium, sodium starch glycolate, alginic acid, corn starch, potato starch, bentonite, methylcellulose, agar and carboxymethylcellulose.

Examples of glidants include, but are not limited to, colloidal silicon dioxide, talc, and corn starch.

Examples of wetting agents include, but are not limited to, propylene glycol monostearate, sorbitan monooleate, diethylene glycol monolaurate and polyoxyethylene laural ether.

Examples of lubricants include magnesium or calcium stearate, sodium lauryl sulphate, talc, starch, lycopodium and stearic acid as well as high molecular weight polyethylene glycols.

Methods for making formulations, for example, suitable for oral administration, are known in the art, such as in Remington: The Science and Practice of Pharmacy, 23rd Ed. (Academic Press), 2020.

### Example 1

To determine whether irdabisant (or pharmaceutically acceptable salt thereof, such as irdabisant hydrochloride) leads to dopamine release in the nucleus accumbens under basal conditions similar to those in which pitolisant, ABT-239 and JNJ-39220675 were tested and found to have no effect, in vivo microdialysis was performed in freely moving rats. Dopamine levels were monitored for 210 minutes following compound administration. Irdabisant hydrochloride was administered intraperitoneally (IP) at 0.001, 0.01, 0.3 and 10 mg/kg for 30 minutes following an IP vehicle (0.5% methylcellulose and 0.2% Tween 80) injection.

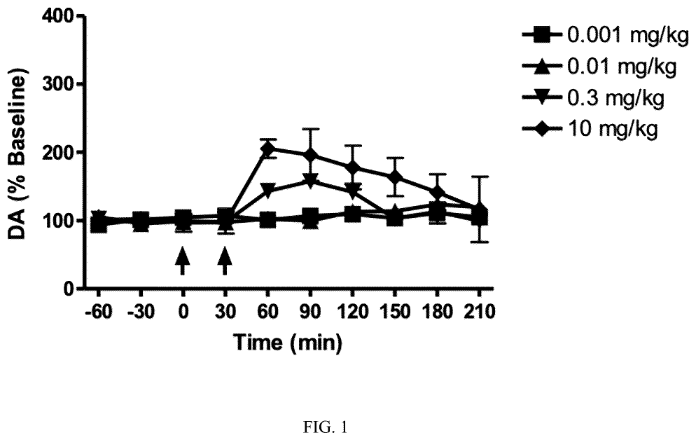

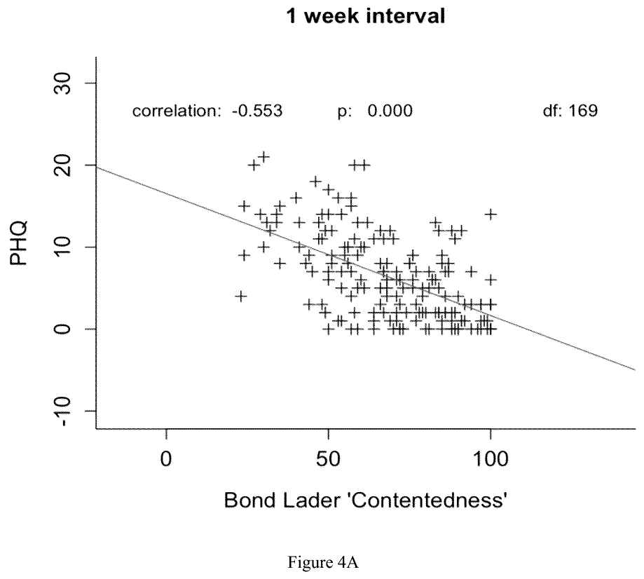

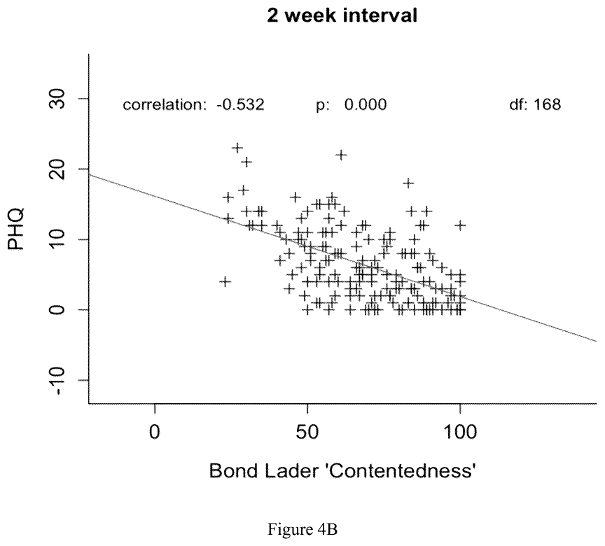

Male Sprague-Dawley albino rats weighing 250 to 350 g were housed two per cage and maintained in a controlled 12:12-h light/dark cycle and under constant temperature at 22° C., with free access to food and water. Rats were anesthetized with the modified Equithesin mixture (see, e.g., Li et al., Neuropsychopharm., 30, 1986-1995, 2005) and mounted in a stereotaxic frame. Stainless guide cannula with a dummy probe were placed and fixed by cranioplastic cement onto the cortex dorsal both to the medial prefrontal cortex (mPFC) and the nucleus accumbens (NAC). Rats received dual probe implantation for the mPFC and NAC (coordinates: A+3.2, L+0.8 (10° C. inclination), V-5.5 mm and A+2.0, L+1.5 to +1.7, V-7.5 mm, respectively, relative to bregma). Three to five days after cannulation, a dialysis probe was implanted into the mPFC and NAC under slight anesthesia with isoflurane. Rats were then housed individually overnight in a dialysis cage and the probes perfused with Dulbecco's phosphate-buffered saline solution with calcium (Sigma, St. Louis, MO) (138 mM NaCl, 8.1 mM Na₂HPO₄, 2.7 mM KCl, 1.5 mM KH₂PO₄, 0.5 mM MgCl₂, 1.2 mM CaCl₂), pH 7.4) at a rate of 0.4 μL/min. On the day of the experiment, the flow was increased to 1.5 μL/min. Sample collection began one hour later, and samples were collected every 30 min. After 60 min of stable baseline, each rat was dosed twice (indicated by arrows in FIG. 1-4); a vehicle dose at t=0 min., followed 30 minutes later by a dose of irdabisant hydrochloride. Dopamine (DA) and acetylcholine (Ach) samples, measured in the absence of cholinesterase inhibition, were monitored with HPLC coupled to mass spectrometry.

### Data Analysis

Data are presented as percentages of baseline from baseline to 180 minutes after treatment. Mean baseline levels (i.e., an average of 60, 30, and 0 minutes before injection) of DA and ACh in the mPFC and NAC were designated as 100%. Non-transformed data were analyzed at each dose by assessing the change relative to the mean baseline using a one-way repeated measures ANOVA followed by a Bonferroni's post-hoc test as implemented in the SigmaPlot version 11.0.0.77 statistical package (Systat Software, Inc., San Jose CA).

### Results

In contrast to previous findings with pitolisant, ABT-239 and JNJ-39220675, increased dopamine release was observed in the nucleus accumben in response to administration of irdabisant hydrochloride. Nucleus accumbens dopamine efflux occurred with a threshold dose of 0.01 mg/kg IP and a maximal effect of 205.6±6.86% over basal at the 10 mg/kg IP dose (FIG. 1). At the 0.01 mg/kg dose, statistically significant elevation in dopamine levels (p=0.005) was achieved at 180 minutes post-dose (overall effect of irdabisant hydrochloride treatment across timepoints on dopamine release: p<0.001). At the 0.3 mg/kg dose, significance (p=0.02) was achieved at the 90 minute time point (overall effect of irdabisant hydrochloride treatment across timepoints on dopamine release: p=0.003). At the 10 mg/kg dose, significance was achieved at the 60 minute (p<0.001), 90 minute (p<0.001), 120 minute (p=0.004) and 150 minute time points (p=0.042; overall effect of irdabisant hydrochloride treatment across timepoints on dopamine release: p<0.001).

### Example 2

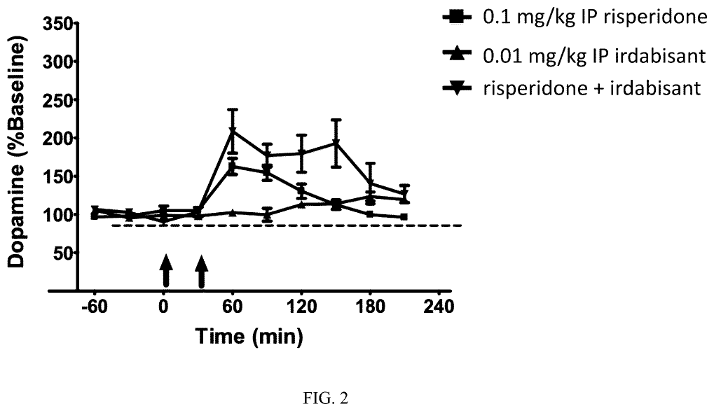

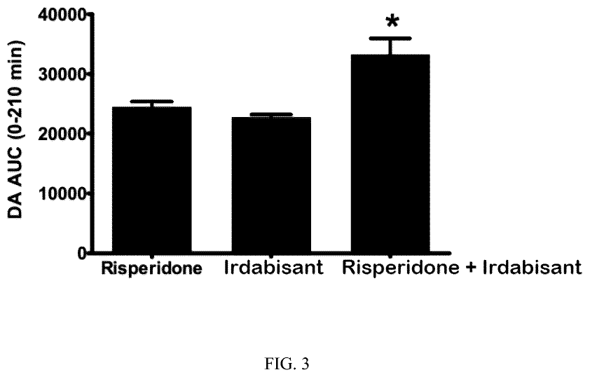

A separate in vivo microdialysis experiment was conducted to determine if the increase in dopamine release in response to administration of irdabisant could be further enhanced in combination with the atypical antispsychotic agent risperidone. Rats were dosed with either 0.01 mg/kg IP irdabisant hydrochloride, 0.1 mg/kg IP risperidone, or a combination of irdabisant hydrochloride and risperidone at the same doses. Area under the curve (AUC) analysis of the total efflux from 0-210 min post-dosing revealed that the combination treatment (0.1 mg/kg IP risperidone+0.01 mg/kg IP irdabisant hydrochloride) produced a significantly greater increase in DA efflux in the nucleus accumbens compared to risperidone (0.1 mg/kg IP) or irdabisant hydrochloride (0.01 mg/kg IP) alone (FIGS. 2 and 3).

These data surprisingly demonstrate that irdabisant, unlike prior reports on multiple other H₃ inverse agonists (pitolisant, ABT-239, and JNJ-39220675) increases dopamine release in the nucleus accumbens under basal conditions. This increase is further enhanced by co-administration with a second generation antipsychotic drug (risperidone). Without being bound by any particular theory, the inventors submit that the effects of irdabisant on reward system dopamine release render it a useful treatment of anhedonia as this is a state of reduced reward system activity as well as dopamine release and signaling.

### Example 3

A study was conducted involving 182 individuals with a range of scores on the Patient Health Questionnaire scale, wherein ≥10 indicates moderate to severe depression (present in 29.1% of participants). Participants completed self-report questionnaires of mood and reward sensitivity, at regular intervals over the course of six weeks (see Table 1 below). All questionnaires and tests were repeated at the final 6 week ‘follow-up’ timepoint. The individuals were also assessed according to the BL-VAS.

TABLE 1
Listing of mood/symptom scales used in this study in addition to BL-VAS.

| Abbreviation | Scale Name | Cadence | Interpretation |
|---|---|---|---|
| PHQ-8 | Patient Health Questionnaire | Every 6 days | Removed the suicidality question |
| | [1] | | from the PHQ-9. Higher scores |
| | | | indicate increased depressive |
| | | | mood/symptoms. |
| SHAPS | Snaith-Hamilton Pleasure | Baseline and | Higher scores indicate greater |
| | Scale [2] | Follow-up | anhedonia |
| DARS | Dimensional Anhedonia | Baseline and | Lower scores indicate greater |
| | Rating Scale [3] | Follow-up | anhedonia. |
| PVSS | Positive Valence Systems | Baseline and | Lower scores indicate greater |
| | Scale [4] | Follow-up | anhedonia. |
| BIS/BAS | Behavioral | Baseline and | Measures sensitivity to reward |
| | Inhibition/Activation System | Follow-up | (BAS) where higher scores show |
| | [5] | | reduced activation; and |
| | | | punishment (BIS) where higher |
| | | | scores show reduced inhibition. |
| TEPS | Temporal Experience of | Every 10 days | Lower scores indicate lower |
| | Pleasure Scale [6] | | pleasure. |
| GAD-7 | General Anxiety Disorder | Baseline and | Higher scores indicate greater |
| | [7] | Follow-up | anxiety |
| PANAS | Positive and Negative Affect | Every (other) 6 | Yields ‘Positive Affect’ (higher |
| | Scale [8] | days | scores are better) and ‘Negative |
| | | | Affect’ scores (lower scores are |
| | | | better). |

The questionnaires described in Table 1 above were all administered at baseline and with repetition at the cadences indicated. The PANAS has been validated over multiple timescales but the version that the inventors have used was targeted to how participants are feeling ‘now’, to provide a closer comparison to the BL-VAS for measuring acute mood. The PHQ asked participants how they have felt over the last two weeks. The TEPS provided a dynamic measure of consummatory pleasure for the study.

TABLE 2
BL-VAS scales show significant correlations at baseline with
most measures of mood, anhedonia, anxiety and symptoms.

| | Domain or | BL-VAS | BL-VAS | BL-VAS | BL-VAS |
|---|---|---|---|---|---|
| Scale | Sub-score | Alertness | Calmness | Contentedness | Total |
| PANAS | Positive Affect | −0.786 | −0.258 | −0.668 | −0.757 |
| | Negative Affect | 0.357 | 0.466 | 0.545 | 0.480 |
| BIS/BAS | Behavioral | 0.262 | 0.262 | 0.254 | 0.289 |
| | Inhibition | | | | |
| | Drive | −0.359 | −0.137 | −0.323 | −0.355 |
| | Fun seeking | −0.203 | −0.004 | −0.203 | −0.198 |
| | Reward | −0.415 | −0.172 | −0.460 | −0.443 |
| | Responsiveness | | | | |
| DARS | Total | 0.419 | 0.262 | 0.440 | 0.452 |
| GAD-7 | Total | −0.445 | −0.428 | −0.553 | −0.532 |
| PHQ | Total | −0.457 | −0.403 | −0.579 | −0.545 |
| PVSS | Effort Valuation | 0.420 | 0.177 | 0.487 | 0.458 |
| | Initial | 0.459 | 0.221 | 0.512 | 0.497 |
| | Responsiveness | | | | |
| | Anticipation | 0.448 | 0.236 | 0.506 | 0.490 |
| | Expectancy | 0.560 | 0.268 | 0.585 | 0.592 |
| | Satiation | 0.380 | 0.162 | 0.506 | 0.439 |
| | Valuation | 0.450 | 0.246 | 0.480 | 0.483 |
| SHAPS | Total | −0.314 | −0.102 | −0.325 | −0.324 |
| TEPS | Anticipatory | 0.481 | 0.316 | 0.552 | 0.538 |
| | Pleasure | | | | |
| | Consummatory | 0.302 | 0.134 | 0.260 | 0.297 |
| | Pleasure | | | | |
| | Consummatory | 0.417 | 0.261 | 0.455 | 0.457 |
| | Context | | | | |

All italicized (57) correlations tabulated are significant with p < .001, except those (5) in bold-italics with p < .01 and (2) bold with p < .05, with 4 associations with underlining as non-significant (p > 0.05).

The BL-VAS scales showed consistent correlations at baseline with nearly all measures (72/76) of mood, motivation, affect, anxiety/depression symptoms and anhedonia (Table 3). The strongest correlations were between BL-VAS Alertness and PANAS Positive Affect (r=−0.79), BL-VAS Total Score and PVSS Expectancy (r=0.59), and BL-VAS Contentedness and PHQ (r=0.58). The correlations with PANAS demonstrate convergent validity for the use of BL-VAS as an immediate measure of affect. The correlations with the PHQ and Expectancy (expectation of enjoyment from future activities) additionally support the BL-VAS as being sensitive to depression and anhedonia symptoms.

TABLE 3
BL-VAS scales show significant correlations with PHQ at 6, 12, 18, 24 and 32 days delay.

| BL-VAS | BL-VAS | BL-VAS | BL-VAS | BL-VAS | | | Duration |
|---|---|---|---|---|---|---|---|
| versus: | Alertness | Calmness | Contentedness | Total | T1 | T2 | days |
| PHQ | 0.457 | 0.403 | 0.579 | 0.545 | Baseline | Baseline | 0 |
| PHQ | 0.376 | 0.381 | 0.553 | 0.483 | Baseline | Visit 3 | 6 |
| PHQ | 0.369 | 0.359 | 0.532 | 0.468 | Baseline | Visit 5 | 12 |
| PHQ | 0.38 | 0.4 | 0.529 | 0.479 | Baseline | Visit 7 | 18 |
| PHQ | 0.301 | 0.349 | 0.486 | 0.413 | Baseline | Visit 9 | 24 |
| PHQ | 0.239 | 0.356 | 0.439 | 0.361 | Baseline | Visit 11 | 32 |
| PHQ | 0.446 | 0.281 | 0.576 | 0.518 | Visit 3 | Visit 5 | 6 |
| PHQ | 0.497 | 0.295 | 0.609 | 0.561 | Visit 3 | Visit 7 | 12 |
| PHQ | 0.41 | 0.317 | 0.554 | 0.489 | Visit 3 | Visit 9 | 18 |
| PHQ | 0.446 | 0.303 | 0.575 | 0.517 | Visit 3 | Visit 11 | 24 |
| PHQ | 0.393 | 0.333 | 0.485 | 0.471 | Visit 5 | Visit 7 | 6 |
| PHQ | 0.357 | 0.304 | 0.45 | 0.435 | Visit 5 | Visit 9 | 12 |
| PHQ | 0.345 | 0.393 | 0.468 | 0.447 | Visit 5 | Visit 11 | 18 |
| PHQ | 0.532 | 0.276 | 0.516 | 0.561 | Visit 7 | Visit 9 | 6 |
| PHQ | 0.59 | 0.302 | 0.537 | 0.603 | Visit 7 | Visit 11 | 12 |
| PHQ | 0.487 | 0.278 | 0.489 | 0.518 | Visit 9 | Visit 11 | 6 |

All italicized (60) correlations tabulated are significant with p < .001, except those (3) in bold-italics with p < .01 and (1) bold with p < .05.

BL-VAS scales are also predictive of PHQ scores taken on the same day as well as with delays ranging from 1 week to more than 4 weeks after the BL-VAS collection. All four scales of the BL-VAS showed predictive validity, with every correlation in Table 3 reaching statistical significance (p<0.05, range r=0.23 to 0.61).

This novel application of the BL-VAS scales shows their ability to use acute, momentary assessments to reliably predict longer-term impacts on mood. The consistent correlations between BL-VAS scores and PHQ scores even beyond four weeks later establish that this immediate measurement tool provides a robust indication of future depressive symptoms. This predictive capacity highlights its utility for early identification of the effectiveness of an intervention for depression. Likewise, the correlation between BL-VAS and other measures of depressive symptoms suggest that change seen with irdabisant over placebo with respect to increasing scores on BL-VAS alertness and contentedness/mood subscales (Baakman et al., Br J Clin Pharmacol., 2019, 85 (5): 970-985, PMID 30710391) are indicative of the antidepressant effects of the drug on a rapid timescale of hours.

TABLE 4
Standard Error of Measurement for BL-
VAS scales across 8 administrations.

| Visit | Total | Alertness | Calmness | Contentedness |
|---|---|---|---|---|
| 1 | 8 | 9.32 | 12.54 | 8.89 |
| 2 | 7.9 | 9.56 | 12.35 | 8.52 |
| 3 | 8.41 | 10.05 | 13.65 | 8.83 |
| 4 | 8.64 | 10.45 | 13.39 | 9.36 |
| 5 | 9.07 | 10.89 | 14.4 | 10.13 |
| 6 | 9.6 | 11.31 | 14.44 | 10.37 |
| 7 | 9.39 | 10.89 | 14.9 | 10.79 |
| 8 | 8.72 | 10.06 | 14.45 | 10.14 |

The standard error of measurement for the four BL-VAS scales are mostly at or below 10 points (Table 4, scales are mean values of comprising items, each on a 0-100 scale). This suggests individual subjects increasing by >=10 points on a 0-100 scale may reflect clinically significant improvement in mood or depression.

### References Cited in Table 1

1. Kroenke, K., Spitzer, R. L., & Williams, J. B. W. (1999). Patient Health Questionnaire-9 (PHQ-9) [Database record]. APA PsycTests.
2. Snaith R P, Hamilton M, Morley S, Humayan A, Hargreaves D, Trigwell P. A scale for the assessment of hedonic tone the Snaith-Hamilton Pleasure Scale. Br J Psychiatry. 1995 July; 167 (1): 99-103.
3. Rizvi S J, Quilty L C, Sproule B A, Cyriac A, Michael Bagby R, Kennedy S H. Development and validation of the Dimensional Anhedonia Rating Scale (DARS) in a community sample and individuals with major depression. Psychiatry Res. 2015 Sep. 30;229 (1-2): 109-19
4. Khazanov, G. K., Ruscio, A. M., & Forbes, C. N. (2020). The Positive Valence Systems Scale: Development and validation. Assessment, 27 (5), 1045-1069
5. Carver, C. S., & White, T. L. (1994). Behavioral inhibition, behavioral activation, and affective responses to impending reward and punishment: The BIS/BAS Scales. Journal of Personality and Social Psychology, 67 (2), 319-333.
6.Chan R C, Shi Y F, Lai M K, Wang Y N, Wang Y, Kring A M. The Temporal Experience of Pleasure Scale (TEPS): exploration and confirmation of factor structure in a healthy Chinese sample. PLOS One. 2012; 7 (4): e35352.
7. Spitzer R L, Kroenke K, Williams J B, Löwe B. A brief measure for assessing generalized anxiety disorder: the GAD-7. Arch Intern Med. 2006 May 22; 166 (10): 1092-7
8. Watson, D., Clark, L. A., & Tellegen, A. (1988). Development and validation of brief measures of positive and negative affect: The PANAS scales. Journal of Personality and Social Psychology, 54 (6), 1063-1070.

### Example 4

Double-Blind, Single and Multiple Dose Study in Patients with Major Depressive Disorder

This study will consist of two sequential double-blind, placebo-controlled treatment periods with two dosages of irdabisant (25 μg and 75 μg) given as a monotherapy, in patients with MDD. The first treatment period (Single-Dose Treatment Period) is a randomized, 3-way crossover design where each participant will receive single oral doses of 25 μg irdabisant, 75 μg irdabisant, and matching placebo given in a random order with a 10-day washout between each dose. The second treatment period (Multi-Dose Treatment Period) begins immediately after Treatment #3, where each participant continues to take their Treatment #3 dose once daily for a total duration of 28 days. The overall purpose of the single dose period is to evaluate patient pharmacodynamic responses to ALTO-203 by measuring cognition and subject experience with the two doses of irdabisant compared to placebo. For the Multi-Dose Treatment Period, the primary objective is evaluation of safety of irdabisant in patients with MDD over 4 weeks. Exploratory objectives will evaluate changes in measures of depression, anhedonia, sleep, and anxiety from Day 1 of Treatment #3 (baseline for Multi-Dose Treatment Period) through the Multi-Dose Treatment Period. In addition, PK properties of the current irdabisant formulation administered in both single and multi-dose regimens will be evaluated.

Approximately 60 to 80 participants will be enrolled to have 60 participants complete all 3 doses in the single dose period.

Irdabisant treatment will be provided as capsules of 25 μg strength and matched placebo. At each dosing, participants will receive a total of 3 capsules, consisting of a combination of active and placebo capsules to make each dose. During the first period of the study, each participant will receive each treatment once in random order. This will be followed by a second treatment period where participants continue once-a-day dosing of their last treatment (Treatment #3) from the single dose period for a total of 28 days.

### Objectives and Endpoints

The study objectives and endpoints are presented in the tables below.

### Single Dose Treatment Period

| | |
|---|---|
| Objective | Endpoint |

Primary

| | |
|---|---|
| To evaluate brain and pharmacodynamic | Results on cognitive tasks and subjective |
| measures in patients with MDD after single | responses (VAS scales) with irdabisant 25 μg |
| dosing with 25 and 75 μg irdabisant compared | and 75 μg compared to placebo. |
| to placebo. | |

Secondary

| | |
|---|---|
| To evaluate safety and tolerability of single | Incidence, severity, and relatedness of TEAEs, |
| doses of 25 and 75 μg irdabisant. | SAEs, discontinuation due to TEAEs, |
| | participant subjective responses, vital signs, |
| | ECG, suicidality, and deaths. |

Exploratory

| | |
|---|---|
| To understand the pharmacokinetics (PK) of | Analysis of plasma concentrations of irdabisant |
| single doses of irdabisant in capsule | after single doses of 25 and 75 μg. |
| formulation. | |
| To understand the association of PK with | Exploration of dose and exposure response |
| pharmacodynamic effects of irdabisant. | characteristics of the pharmacodynamic effects |
| | of irdabisant. |

### Multi-Dose Treatment Period

| | |
|---|---|
| Objective | Endpoint |

Primary

| | |
|---|---|
| To evaluate safety and tolerability of multi- | Incidence, severity, and relatedness of TEAEs, |
| dosing of 25 and 75 μg irdabisant. | SAEs, discontinuation due to TEAEs, |
| | participant subjective responses, vital signs, |
| | ECG, clinical labs, suicidality, and deaths. |

Secondary

| | |
|---|---|
| To understand the pharmacokinetics (PK) of | Analysis of PK sample with up to 28 days of |
| multi-doses of Irdabisant in capsule | dosing and PK modeling. |
| formulation. | |

Exploratory

| | |
|---|---|
| To understand the effects of irdabisant on | Change in measures of depression, anhedonia, |
| depressed mood, symptoms of anhedonia, sleep, | sleep, and anxiety from Day 1 of Treatment #3 |
| and anxiety. | to 4 weeks of treatment with irdabisant |
| | compared to placebo based on MADRS, PHQ- |
| | 9, SHAPS, DARS, PVSS, ISI, CPFQ, and |
| | GAD-7. |
| To relate irdabisant effects to behavior (activity | Change in sleep parameters and activity level of |
| and sleep) derived from wearable device signals | participant with irdabisant compared to placebo |
| and subjective experience. | over 28 days of treatment. |
| evaluate brain and pharmacodynamic measures | Performance on cognitive battery tasks and |
| in patients with MDD with | subjective responses (VAS scales) after |
| multiple doses of 25 and 75 μg irdabisant | multiple doses of irdabisant 25 μg and 75 μg |
| compared to placebo. | compared to placebo. |
| To understand the association of PK with | Exploration of dose and exposure response |
| pharmacodynamic effects of irdabisant. | characteristics of the pharmacodynamic effects |
| | of irdabisant. |

This study is designed to determine the pharmacodynamic effects of irdabisant in MDD as well as assess its safety, tolerability, and PK in patients with MDD over 28 days. Specifically, irdabisant has demonstrated the ability to promote positive subjective experiences and cognitive effects in healthy participants after a single dose. These effects will be tested in patients with MDD to determine the potential of irdabisant as an antidepressant. Two doses of irdabisant (25 and 75 μg) will be compared to placebo after single dose administration as well as after multiple daily doses. The behavioral battery is designed to capture an array of relevant mental functions including aspects of cognition and reward sensitivity, which are relevant for patients with MDD, including anhedonia symptoms. Subjective responses to irdabisant (across a range of mood and emotional factors) will be captured by VAS scales. Exploratory measures will include evaluation of impact of treatment over 28 days on depressed mood assessed using change from pre-treatment assessments (Visit 1 or 2 depending on the specific test) on the Montgomery-Åsberg Depression Rating Scale [MADRS]), anhedonia (assessed using the Dimensional Anhedonia Rating Scale [DARS], the Snaith Hamilton Pleasure Scale [SHAPS], and the Positive Valence Systems Scale [PVSS]), sleep (assessed using the insomnia severity scale [ISI]) and anxiety (assessed using the 7-item Generalized Anxiety Disorder Scale [GAD-7]). Objective measures of sleep will be captured by a wearable device.

The study will consist of two treatment periods. The initial Single-Dose Treatment Period is a randomized, double-blind, placebo-controlled 3-way crossover design with oral administration of single doses of irdabisant 25 and 75 μg doses, as well as matching placebo. For each treatment in this period, participants will complete a battery of brain-behavioral tests including VAS scales around Tₘₐₓ of irdabisant, approximately 5 hours after dosing. With the single dose crossover design, each participant serves as their own control, increasing the power for the analyses to reduce the inter-subject variability. Participants and evaluating staff will be blinded to the dosing condition. Effects with single doses of irdabisant on cognitive measures and VAS scales have been seen in previous studies in healthy controls as well as with another H₃R AIA in a study conducted by the Sponsor.

In the Multi-Dose Treatment Period, participants will continue once-daily dosing with the last treatment (Treatment #3) received in the prior period for a total of 28 days. Safety, PK parameters, and additional exploratory PD effects will be evaluated during this 4-week monotherapy phase. Measures of depression, anxiety, sleep, and anhedonia will also be collected during this 4-week period.

### Inclusion Criteria

In order to be eligible to participate in this study, an individual must meet all of the following criteria:

Adult, aged 25 to ≤64 years old at the time of signing the informed consent form (ICF).

Have a diagnosis of MDD based on the SCID-5 and DSM-5 depression criteria items, with a score of ≥10 on the PHQ-9 at each of Visits 1 and 2. Have moderate MDD as confirmed by MADRS total score between 20-36 at Visit 1.

Presence of anhedonia symptoms with a score of ≥28 on the Snaith Hamilton Pleasure Scale (SHAPs).

Based on clinical assessment have either:
had a period of remission of at least 2 consecutive months (not meeting DSM-5 criteria for MDD) in the past 26 months, regardless of the number of failed antidepressants OR
did not have a period of remission of at least 2 consecutive months in the past 26 months and within the past 24 months have not failed >3 antidepressants (including current antidepressant) at an adequate dosage and duration as defined by the ATRQ.

At Screening Visit 2 is not taking any antidepressant medication. Patients may be treatment-naïve at screening. Previous use of an antidepressant in this episode or prior episode of MDD is allowed. Otherwise, patients taking an oral antidepressant at screening may be eligible to enter the study if per clinical judgement, and with patient agreement, it is considered clinically appropriate to discontinue current treatment at least 5 days prior to Visit 2.

Medically healthy (to the PI's or designee's discretion) without clinically significant abnormalities at Screening Visits 1 or 2, including:
a. Physical examination, vital signs including temperature, heart rate (HR), respiratory rate, and blood pressure (BP).
b. Calculated creatinine clearance (CrCl) or glomerular filtration rate (GFR)>50 mL/min using CKD-EPI equation; alanine transaminase (ALT) and aspartate transaminase (AST) ≤2×the upper limit of normal (ULN); results of all other clinical chemistry without any clinically significant (CS) abnormality.

Agree to assessments (neurocognitive testing, subjective questionnaires, and activity and sleep monitoring by wearable device) and willing to comply with all study procedures, able to complete all assessments independently, and available for the duration of the study.

Body mass index (BMI)≥18.0 and ≤35.0 kg/m² (inclusive).

Females who are not pregnant, breastfeeding or planning to get pregnant or to donate eggs until at least 30 days after the last study treatment. Males must not father a child or donate sperm for the duration of the study, and for 90 days following final study treatment dose.

### Exclusion Criteria

Participants are excluded from the study if any of the following criteria apply:

Diagnosis of bipolar disorder or a psychotic disorder based on the SCID for DSM-5.

Diagnosis of dementia based on history or clinical assessment.

Significant, current PTSD symptoms based on SCID diagnosis or history of PTSD and PCL-5>50 at Visit 1.

Concurrent use of any of the following at Screening Visit 2:
a) Antidepressant medication
b) Mood stabilizers (anticonvulsants for non-psychiatric reasons may be allowed with Sponsor's permission)
c) Antipsychotic medications
d) Benzodiazepines, stimulants, or opiate pain medications greater than 3 days per week and unable to reduce use to 3 or fewer days per week on an as needed basis. These medications may not be used for 24 hours before a biomarker assessment.

Note: hypnotics (such as GABAergic Z-drugs, orexin antagonists, and sedating antihistamines) may be used on a daily basis if a participant enters the trial (at Visit 1) taking a hypnotic on a nightly basis. As needed use of hypnotics is allowed up to 3 days per week but not for 24 hours before biomarker assessments.

Note: Medications may be modified to meet inclusion criteria if clinically indicated. Medications should be discontinued long enough before Visit 2 (at least 5 days or 5 half-lives (5×t1/2), whichever is longer) to ensure no withdrawal symptoms are present.

Have received electroconvulsive therapy (ECT), deep brain stimulation (DBS), vagus nerve stimulation (VNS), >2 treatments with ketamine or esketamine in the current depressive episode (a new depressive episode occurs if someone does not meet DSM-5 criteria for MDD for two consecutive months).

Unstable psychotherapy as defined by a change in frequency and/or type of therapy in the 6 weeks prior to Visit 2 if the frequency is ≤2×/month or in the last 3 months if the therapy is >2×/month (approximately weekly) prior to Visit 2

Any of the following medical conditions:
a) Any uncontrolled medical condition or history of significant cardiovascular, pulmonary, hepatic, renal, hematological, gastrointestinal, endocrine, immunologic, dermatologic, or neurological disease, including any acute illness or major surgery within the past 3 months determined by the PI or designee to be clinically significant. An unstable medical condition may be indicated by a medical hospitalization or a change in medications for a chronic medical condition in the last 3 months.
b) History of seizure. Childhood febrile seizure is not exclusionary.
c) Severe impediment to vision, hearing, comprehension, and/or hand movement that interferes with study tasks.
d) Any clinically significant screening laboratory results (labs may be repeated once), in the opinion of the investigator.
e) Personal history of QT prolongation, ventricular arrhythmias, ischemic heart disease, or cardiomyopathy or family history of sudden cardiac death.
f) Clinically significant ECG during screening including QTcF>450 msec for males and >470 msec females.
g) Blood pressure (BP)>150 systolic or >95 diastolic while sitting at rest for 5 minutes.

Caffeine or coffee/tea consumption ≥the equivalent of 5 or more standard drinks of coffee per day (or approximately 500 mg caffeine or more).

History of suicide attempts in the past 6 months, or a CHRT-SR12 score of 3 or 4 for suicidal ideation (items 10, 11, or 12) during screening (Visits 2 and 3) or investigator assessment based on results from the CHRT-C and clinical assessment during screening or prior to dosing.

Receipt of any other CNS investigational medication or investigational device within 6 months prior to Screening Visit 2.

Prior exposure to irdabisant.

Positive urine toxicology (not including marijuana or documented prescription). One retest during screening is allowed. Current moderate or severe substance use disorder (SUD) (other than nicotine) determined by clinical assessment is exclusionary. Participants with a history of SUDs need to be in remission for at least 3 months prior to Visit 1.

Use of macro-doses of psychedelics in the 6 months prior to Visit 1 or microdosing for 6 weeks prior to Visit 2 (please discuss with the Sponsor if not sure if macro-or microdosing).

Excessive use of alcohol defined by (on average)>14 standard drinks/week for males and >7 standard drinks/week for females.

Positive alcohol test at Screening Visit 1 or Day 1 of the single dose period.

History of lactose intolerance.

Known history of anaphylaxis to any medication.

Lymphoma, leukemia, or any malignancy within the past 5 years; localized basal cell or squamous epithelial carcinomas of the skin is not exclusionary.

Any other condition and/or situation that the Investigator believes may interfere with the safety of the participant, study conduct, or interpretation of study data.

All references (including patent publications) cited herein are hereby incorporated by reference.

1. A method of treating a patient having a psychiatric or neurological disorder with anhedonia comprising administering to the patient an effective amount of irdabisant or a pharmaceutically acceptable salt thereof as the sole active agent.

2. A method of treating a patient having a psychiatric or neurological disorder with anhedonia comprising administering to the patient an effective amount of (a) irdabisant or a pharmaceutically acceptable salt thereof, and (b) an antipsychotic agent.

3. The method of claim 1, wherein the psychiatric or neurological disorder is major depressive disorder, bipolar depression, post-traumatic stress disorder, substance use disorder, anhedonia-related aspects of schizophrenia, schizoaffective disorder, or any combination of any of the foregoing.

4. The method of claim 1, wherein the psychiatric or neurological disorder is bipolar I disorder or bipolar II disorder.

5. The method of claim 1, wherein the psychiatric or neurological disorder is negative symptoms of schizophrenia.

6. The method of claim 1, wherein the psychiatric or neurological disorder is motivational deficits or anhedonia in schizophrenia.

7. The method of claim 1, wherein the psychiatric or neurological disorder is depression.

8. A method of treating anhedonia in a patient comprising administering to the patient an effective amount of irdabisant or a pharmaceutically acceptable salt thereof as the sole active agent or in combination with an antipsychotic agent.

9. A method of treating anhedonia in a patient having major depressive disorder comprising administering to the patient an effective amount of irdabisant or a pharmaceutically acceptable salt thereof as the sole active agent or in combination with an antipsychotic agent, antidepressant, or any combination of any of the foregoing.

10. The method of claim 2, wherein the antipsychotic agent is a typical (first generation) antipsychotic, an atypical (second generation) antipsychotic, a third generation (D2 partial agonist) antipsychotic, or any combination of any of the foregoing.

11. The method of claim 10, wherein the antipsychotic agent is a typical (first generation) antipsychotic.

12. The method of claim 11, wherein the typical (first generation) antipsychotic is benperidol, bromperidol, droperidol, haloperidol, moperone, fluspirilene, penfluridol, pimozide, acepromazine, chlorpromazine, cyamemazine, dixyrazine, fluphenazine, levomepromazine, mesoridazine, perazine, periciazine, perphenazine, peoptiazine, prochlorperazine, promethazine, prothipendyl, thioproperazine, trifluoperazine, chlorprothixene, clopenthixol, flupentixol, thiothixene, zuclopenthixol, a pharmaceutically acceptable salt of any of the foregoing, or any combination of any of the foregoing.

13. The method of claim 10, wherein the antipsychotic agent is an atypical (second generation) antipsychotic.

14. The method of claim 13, wherein the atypical (second generation) antipsychotic agent is amisulpride, nemonapride, remoxipride, sultopride, lumateperone, lloperidone, paliperidone, perospirone, risperidone, ziprasidone, lurasidone, melperone, lumateperone, asenapine, clozapine, olanzapine, quetiapine, zotepine, blonanserin, pimavanserin, sertindole, a pharmaceutically acceptable salt of any of the foregoing, or any combination of any of the foregoing.

15. The method of claim 10, wherein the antipsychotic agent is a third generation (D2 partial agonist) antipsychotic.

16. The method of claim 15, wherein the third generation antipsychotic agent is aripiprazole, brexpiprazole, cariprazine, brilaroxazine, a pharmaceutically acceptable salt of any of the foregoing, or any combination of any of the foregoing.

17. The method of claim 2, wherein the antipsychotic agent is risperidone, aripiprazole, brexpiprazole, amisulpride, olanzapine, quetiapine or a pharmaceutically acceptable salt thereof.

18. The method of claim 17, wherein the antipsychotic agent is risperidone or a pharmaceutically acceptable salt thereof.

19. The method of claim 1, wherein the patient exhibits a reduction according to the Bond-Lader Visual Analogue Scale (BL-VAS).

20. The method of claim 1, wherein about 1 μg to about 500 μg per day of irdabisant or a pharmaceutically acceptable salt thereof (based on the equivalent amount of irdabisant hydrochloride) is orally administered.

21. The method of claim 1, wherein about 5 μg to about 250 μg per day of irdabisant or a pharmaceutically acceptable salt thereof (based on the equivalent amount of irdabisant hydrochloride) is orally administered.

22. The method of claim 1, wherein the irdabisant or a pharmaceutically acceptable salt thereof is irdabisant hydrochloride.

23. The method of claim 1, wherein about 25 μg of irdabisant hydrochloride is orally administered once daily.

24. The method of claim 1, wherein about 75 μg of irdabisant hydrochloride is orally administered once daily.

25. The method of claim 1, wherein the irdabisant or a pharmaceutically acceptable salt thereof is orally administered.

26. The method of claim 18, wherein the dose of risperidone or a pharmaceutically acceptable salt thereof (based on the equivalent amount of risperidone free base) ranges from about 0.25 mg to about 16 mg per day.

27. The method of claim 26, wherein the dose of risperidone or a pharmaceutically acceptable salt thereof (based on the equivalent amount of risperidone free base) ranges from about 1 mg to about 8 mg per day.

28. The method of claim 2, wherein the antipsychotic agent is orally administered.

29. A method for evaluating a drug for the treatment of depression (such as major depressive disorder) comprising, for each patient,
(a) evaluating the patient prior to initiation of treatment with the drug using the BL-VAS,
(b) initiating treatment of the patient with the drug, and
(c) at least once evaluating the patient during treatment with the drug using the BL-VAS, wherein the drug is considered effective for depression if there is an improvement in the BL-VAS for the patients tested.

30. The method of claim 29, wherein the drug is considered effective for depression if there is an improvement for the patients tested in the BL-VAS total score, BL-VAS alertness sub-score, BL-VAS calmness sub-score, BL-VAS contentedness, or any combination of any of the foregoing.
